(19) United States
(12) Patent Application Publication
(10) Pub. No.: US 2003/0215811 A1
(43) Pub. Date: Nov. 20, 2003

(54) Detection of microsporidial species using a quantitative real-time PCR assay

(76) Inventors: Frank Schaefer, Sharonville, OH (US); H.D. Alan Lindquist, Cincinnati, OH (US); Jeffery Dean Hester, Cincinnati, OH (US); Manju Varma, Cincinnati, OH (US)

Correspondence Address: BROWDY AND NEIMARK, P.L.L.C., 624 NINTH STREET, NW, SUITE 300, WASHINGTON, DC, 20001-5303, US

(21) Appl. No.: 10/147,322

(22) Filed: May 17, 2002

(51) Int. Cl. C12Q 1/68; C12P 19/34

(52) U.S. Cl. 435/6; 435/91.2

(57) ABSTRACT

Microsporidial species can be detected in samples using real-time PCR dual-fluorescent assays with species-specific primer set and a dual fluorescent labeled hybridization probe. Cyclospora cayetanensis can be detected in samples using a real-time PCR dual-fluorescent assay with a primer set and dual fluorescent labeled hybridization probe.

### FIELD OF THE INVENTION

The present invention relates to real-time PCR assays for detecting parasites.

### BACKGROUND OF THE INVENTION

A number of parasites are etiological agents for diseases in humans, several of which are described below. Cyclospora cayetanensis, a coccidian parasite of humans, has been recognized worldwide as an emerging pathogen in both immunocompromised (Ortega et al., 1993) and immunocompetent individuals (Berlin et al., 1994). Presently, humans appear to be the primary host for this parasite (Eberhard et al., 2000). Clinical manifestations associated with C. cayetanensis can include prolonged diarrhea, nausea, abdominal cramps, anorexia, weight loss, and other symptoms of gastroenteritis. The transmission form of C. cayetanensis is an environmentally resistant, 8-10 micron spherical oocyst that contains two ovid sporocysts, each of which contains two sporozoites. Cyclospora cayetanensis oocysts shed in the feces of an infected host are not infectious until they become sporulated. Depending upon a variety of environmental factors such as temperature and humidity, sporulation occurs after approximately two weeks outside the host. A striking feature of this parasite is its seasonality. In some areas, during the rainy season, infection rates are high, decreasing to undetectable levels during the dry season. While the mode of transmission has not been completely elucidated, most cases of cylsporiasis in the U.S. have been associated with consumption of fruits and vegetables that may have become contaminated after contact with tainted water (Berlin et al., 1994; Herwaldt et al., 1997). Since the epidemiology of this disease is still in doubt, it would be valuable to have sensitive and specific tools to detect and quantify the presence of this organism in the environment. These tools might also lead to the development of more sensitive and specific clinical diagnostic methods.

Other parasites which are recognized as etiological agents of disease in humans include the microsporidial species Encephalitozoon hellem, Encephalitozoon cuniculi, and Encephalitozoon intestinalis. These three obligate intracellular protozoan parasites belong to the phylum Microsporidia, which includes nearly 150 genera with more than 1000 species. Members of this phylum form uniquely diagnostic, environmentally resistant spores that infect a wide range of invertebrates and all classes of vertebrates. (Curry et al., 1993; Didier et al., 1998; Sprague et al., 1992). The transmission form of these three Encephalitozoon species is an ovid to pyriform shaped spore measuring approximately 1.0-1.5 microns wide and 2.0-2.5 microns long. Infections caused by these three microsporidial species have predominantly been recognized with the immunologically compromised, including transplant patients (Latib et al., 2001) and those with acquired immune deficiency syndrome (AIDS) (Didier et al., 1996; De Groote et al., 1995; Frazer et al., 1995). Recently, these pathogens have also been identified in associated with immunocompetent populations (van Gool et al., 1997; Enriquez et al., 1998) and travelers (Raynaud et al., 1998). Because microsporidia are ubiquitous in their geographical distribution, it is not surprising that the prevalence of microsporidiosis in studies of patients with chronic diarrhea ranges from 7 to 50% worldwide (Bryan, 1995; Muiller et al., 2001).

While over the last ten years the presence of human-pathogenic microsporidia has routinely been reported in clinical specimens, their existence within environmental samples has only recently been described (Cotte et al., 1999; Dowd et al., 1998). Detecting human-pathogenic microsporidia in surface water is not surprising, with such a large population of individual shedding infectious spores via body fluids (urine and respiratory secretions) and feces. While the water industry has become increasingly concerned, and numerous studies have been performed, only a small amount of reported data confirm that human-pathogenic microsporidia are present in surface water (Sparfel et al., 1997). The limited amount of evidence for microsporidial waterborne reservoirs can in part be attributed to the need for specific, sensitive and practical methods to identify these small protozoan pathogens to the species level in a matrix as complex as environmental water.

Currently diagnostic procedures used for the routine detection of microsporidia in clinical samples are non-selective histochemical stains such as the modified trichrome stain and fluorescent brighteners (Ignatius et al., 1997). While staining techniques in conjunction with light or epifluorescence microscopy usually allow for the detection of microsporidial spores in clinical specimens, sensitivity is low, and species differentiation is nearly impossible. Furthermore, this technique cannot readily be adapted for routine testing of environmental samples because environmental water concentrates can be expected to contain very low numbers of human-pathogenic microsporidia located within a very complex matrix that also contains microsporidial species that have not been associated with human pathogenicity. While other techniques that use fluorescent-labeled monoclonal antibodies (Moura et al., 1999), fluorescent in situ hybridization (FISH) probes (Hester et al., 2000), or DNA sequencing (Ombrouck et al., 1996) have been reported, they are not routinely used for detecting Encephalitozoon species in either clinical or environmental samples. Because of the low sensitivity of immunofluorescence procedures, time-consuming and labor intensive multi-step FISH probing protocols and false positives PCR-amplicon carry-over, the results obtained from these techniques are not consistently reliable.

Conventional detection of microsporidia in clinical samples is typically performed by direct visualization of spores using light microscopy. However, detecting these parasites even using histochemical staining methods is not a trivial task, because of the small size and non-distinct shape of the spores. Because of the small size of microsporidial spores and the inherent complexity of the clinical and environmental matrices in which they are located, alternative molecular based detection methods have been found to be advantageous. PCR amplification coupled with either agarose-gel electrophoresis or DNA sequencing has proven to be sensitive for detecting Encephalitozoon microsporidial species. However, there are problems with false positives because of PCR amplicon carry-over and the inability to obtain accurate quantitative data which makes it impractical to use these techniques in routine clinical and environmental assays. False positives in a clinical setting would lead to inaccurate diagnoses and institution of unneeded or inappropriate therapeutic strategies. Furthermore, accurate quantitative data are desperately needed to assess the prevalence of human-pathogenic microsporidia in environmental water sources to develop appropriate risk assessment strategies and effective risk management practices.

More specifically, C. cayetanensis is currently detected using microscopic examination of the autofluorescence (Berlin et al., 1998) and staining characteristics of the oocysts (Clarke et al., 1996), and molecular identification by either spectrophotometric based detection of an oligonucleotide-ligation assay (Jinneman et al., 1991) or polymerase chain reaction (PCR) in combination with agarose gel electrophoresis (Jinneman et al., 1998; Orlandi et al., 2000; Pieniazek et al., 1996; Sturbaum et al., 1998). The variability in autofluorescence and staining precludes microscopic analysis for routine detection of this organism. Even through spectrophotometric based detection of an oligonucleotide-ligation assay and PCR in combination with agarose gel electrophoreses have shown great promise with respect to species-specific identification of C. cayetanensis, these techniques are time-consuming, labor intensive, and subject to problems from contamination.

Therefore, there is a need to overcome the shortcomings of the current techniques by developing a quantitative detection method for pathogens that is sensitive, specific, and amenable for use in routine processing of large numbers of clinical or environmental samples.

### SUMMARY OF THE INVENTION

It is an object of the present invention to overcome the aforesaid deficiencies in the prior art.

It is another object of the present invention to provide a method for detecting pathogens in clinical and environmental samples.

It is yet another object of the present invention to provide a method for detecting Encephalitozoon species and Cyclospora species in clinical and environmental samples.

The present invention provides real-time PCR assays for specific pathogens in environmental and clinical samples.

The principles of fluorogenic 5′ nuclease PCR assays has been described extensively (Foy et al., 2001), and has been successful in accurately quantifying protozoa (Higgins et al., 2001; Lin et al., 2000), fungi (Haugland et al., 1999; Roe et al., 2001; Vesper et al., 2000), bacteria (Hardegger et al., 2000; Makino et al., 2001; McAvin et al., 2001; Wellenghausen et al., 2001), and viruses (Chen et al., 2001; Dehee et al., 20011 Jardi et al., 2001; Verstrepen et al., 2001) in an extensive number of environmental and clinical matrices. The fluorogenic 5′ nuclease PCR assay of the present invention facilitates quantitative species-specific identification of a variety of spores of pathogens in both clinical and environmental samples.

The process of the present invention provides assays suitable for rapid, sensitive, quantitative, high-throughput detection of human pathogenic microsporidial species, particularly Encephalitozoon and Cyclospora species. The Encephalitozoon assays use species-specific primer sets and a genus-specific dual fluorescent-labeled probe that anneal to a region within the Encephalitozoon 16S rRNA gene. The assay design permits the probe to be used either with one set of primers for a level-level determination, or with a combination of primer sets for a genus-level screening of samples. For detection of Cyclospora species, the assay uses a species-specific primer set and dual fluorescent labeled C. cayetanensis hybridization probe using a unique genetic sequence within the 18S ribosomal ribonucleic acid gene sequence of Cyclospora cayetanensis to distinguish it from other pathogenic protozoa. This real-time polymerase chain reaction assay has been optimized to specifically detect a single Cyclospora oocyst.

According to the present invention, the 5′-exonuclease activity of DNA polymerase is used to cleave a species-specific DNA hybridization probe. This DNA hybridization probe anneals between a set of species-specific primers during the anneal-extension step of PCR. The probe is dual-labeled with a reporter fluorescent dye on the 5′ end, and a quencher florescent dye on the 3′ end. In addition, the 3′ end of the probe is phosphorylated to prevent extension of the probe during PCR. Prior to cleavage by polymerase, the proximity of the fluorescent dyes causes fluorescent resonance energy transfer (FRET) to occur. Thus, the emission spectrum of the reporter fluorescent dye is quenched by the second fluorescent dye. However, then the fluorescently labeled DNA hybridization probe is annealed between the species-specific primer set and primer elongation occurs. The probe is cleaved by the DNA polymerase, releasing the fluorescent moiety from the probe. This, in turn, interrupts the fluorescent resonance energy transfer, and a sequence-specific signal is generated. The amount of fluorescent signal generated during the anneal-extension step of a PCR cycle within this fluorogenic probe-based PCR assay is directly proportional to the amount of specific amplicon produced. The cycle at which the increase in fluorescence of a sample exceeds the background is called the threshold cycle (C_{T}). The C_{T} value is inversely proportional to the starting number of DNA template molecules. The higher the initial DNA template copy number, the lower the threshold cycle measured. By comparing the C_{T} value to a calibration curve constructed with known amounts of DNA, the starting number of DNA template molecules in an unknown sample can be determined.

### BRIEF DESCRIPTION OF THE DRAWINGS

FIG. 1 shows the relative position of the selected probe and primer set with respect to the 18S rRNA gene sequence of C. cayetanensis.

FIG. 2 shows typical amplification plots (ΔRₙ vs. PCR cycle) corresponding to 10³-10⁻¹ C. cayetanensis oocysts.

FIG. 3 is a calibration curve showing C_{T} values versus serial dilutions of C. cayetanensis DNA, ranging from 10³-10⁰ oocysts.

FIG. 4 is a diagrammatic representation of the fluorogenic 5′nuclease PCR assays according to the present invention. The nucleotide positions are based on the 16S rRNA gene sequence of (a) Encephalitozoon cuniculi (GenBank accession No. X98469), (b) Encephalitozoon hellem (GenBank accession No. L39108) and (c) Encephalitozoon intestinalis (GenBank accession No. U09929).

FIG. 5 is a melting curve analysis of species-specific PCR amplicons of Encephalitozoon hellem, Encephalitozoon intestinalis, and Encephalitozoon cuniculi using the SYB Green double-stranded DNA binding dye. The Encephalitozoon hellem, Encephalitozoon intestinalis, and Encephalitozoon cuniculi-specific PCR amplicons had characteristic melting temperatures that depend on their GC content.

FIG. 6 is a typical amplification plot (AR vs. PCR cycle) constructed using extension phase fluorescent emission data collected during PCR amplification during the fluorogenic 5′; nuclease PCR assay. Serial ten-fold dilutions of DNA corresponding to 10⁴-10⁻¹ Encephalitozoon hellem spores were used. The non-template control that contains reagent water rather than DNA template is not discernible from background.

FIG. 7a shows sensitivity and detection range of the fluorogenic 5′nuclease assays for Encephalitozoon cuniculi.

FIG. 7b shows sensitivity and detection range of the fluorogenic 5′nuclease assays for Encephalitozoon hellem.

FIG. 7c shows sensitivity and detection range of the fluorogenic 5′nuclease assays for Encephalitozoon intestinalis.

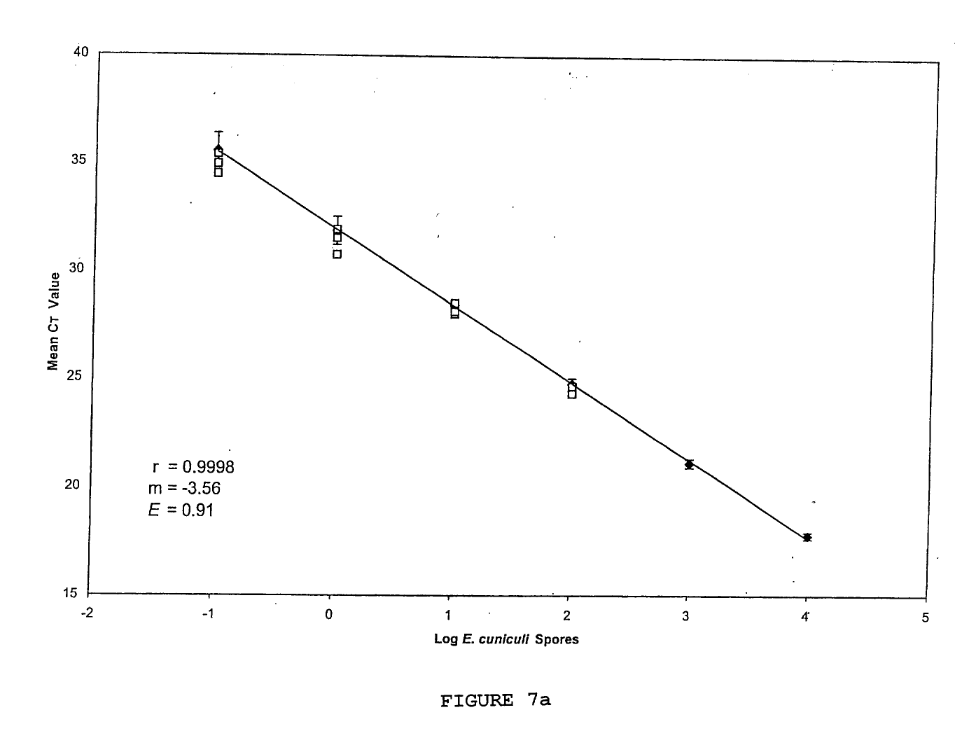

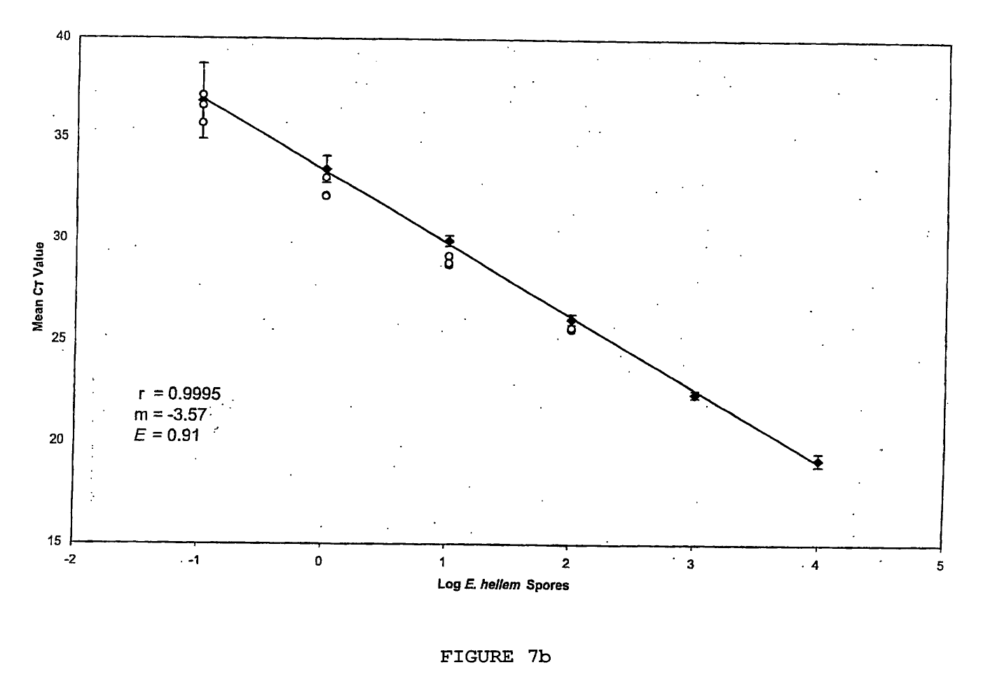

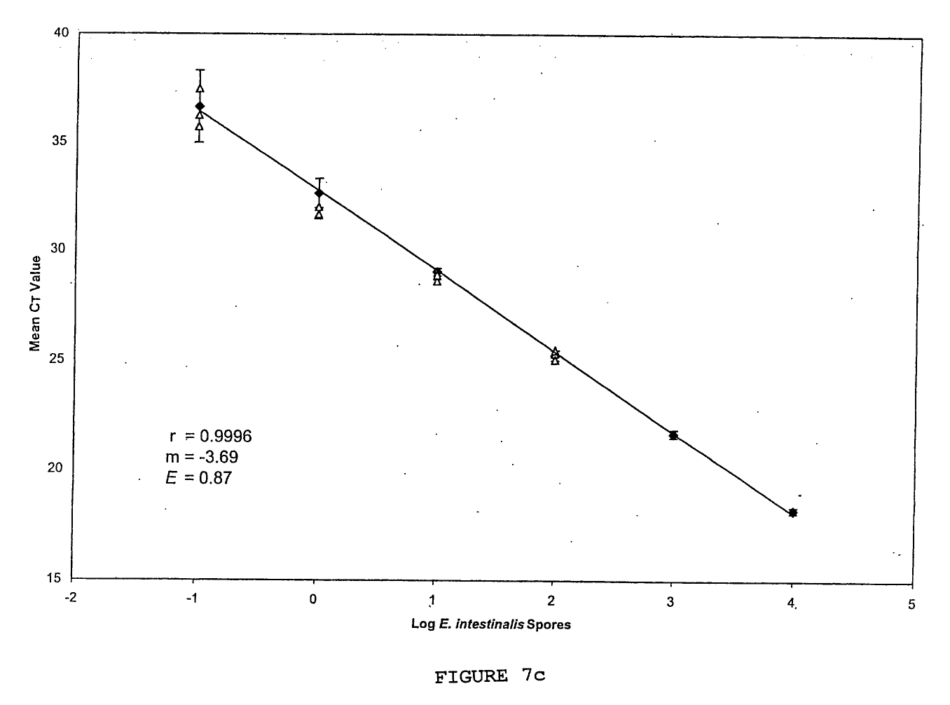

The calibration curves for FIGS. 7a-7c were generated using C_{T} values obtained from serial 10-fold dilutions of genomic DNA that would correspond to 104-10-1 hemacytometer counted spores. Each solid data point corresponds to the mean C_{T} value of 15 replicates, five from each of the three individual DNA extraction procedures. The standard deviations of the data points used in the calibration curves are shown with error bars. The correlation coefficients (r), slopes (m), and PCR amplification efficiencies (E=10⁻¹/m⁻¹) for each calibration curve are given. Non-solid data points correspond to DNA isolated from flow cytometric enumerated spores (10²-10⁻¹) that was used to validate the calibration curves. Each non-solid data point corresponds to the mean CT value of 15 replicates, five from each of the three individual DNA extraction procedures.

### DETAILED DESCRIPTION OF THE INVENTION

The present invention provides 5′ nuclease fluorogenic PCR assays for quantitative species-specific identification of oocysts or spores in both clinical and environmental samples. This technique makes it possible to detect parasites in a wide variety of food, clinical, and environmental samples, as it is robust, reliable, and sensitive.

Methods

Parasites

Cyclospora cayetanensis oocysts were purified from potassium dichromate preserved fecal specimens using a sieving and sucrose flotation procedure.

Enumeration of Oocysts

Purified Cyclospora cayetanensis oocysts were enumerated using both a flow cytometer and hemacytoemter.

Flow cytometry: Flow cytometric counting of Cyclospora cayetanensis oocysts was performed using a FACSVantaqe SE (Becton Dickinson, San Jose, Calif.) equipped with clone-Cyt software (Beckton Dickinson). The cytometer was equipped with a 488 nm argon laser and the sheath fluid used was FACSFlow (Becton Dickinson). The oocysts were gated by forward (FSC), side (SSC), scatter and FL5. A primary gate was set by FSC and SSC. The secondary gate was FSC and FL5. The settings used were FSC gain 1, FSC threshold 236V; SSXC gain 1, 236 V; and FL5 log gain V635. Five replicates of oocysts suspensions of 8000, 800, and 80 were sorted into 1.5 ml centrifuge tubes containing 0.1 ml reagent water.

Hemacytometer: The Cyclospora cayetanensis oocyst stock suspension concentration was determined using a hemacytometer and a Zeiss Axioskop 20 microscope (Carl Zeiss, Oberkochen, Germany) equipped with bright field optics at 400X magnification. For an accurate assessment of the oocyst concentration, six hemacytometer platforms were counted.

Real-Time PCR Probe and Primer Sets for Cyclospora cayetanensis

The Cyclospora cayetanensis-specific Taqman probe and primer set used in this example were designed using the 18S rRNA gene sequences of Cyclospora cayetanensis and other closely related species in the family Eimeriida. The 18S rRNA gene sequences were aligned using the MEGALIGN software package (DNASTAR, Inc., Madison, Wis.). Unique regions within the 18S rRNA gene sequence of Cyclospora cayetanensis were identified as possible target sites for the Taqman probe and primer set. The Taqman probe and primer set were selected using the ABI Primer Express® program (Applied Biosystems, Foster City, Calif.) according to the manufacturer's instructions. To confirm species specificity, the nucleic acid sequences of the Cyclospora cayetanensis-specific probe and primer sets were checked against all available nucleic acid sequences in the NCBI GenBank database using the Blast Search program. Synthesized Taqman probes and primer sets were purchased from Applied Biosystems.

Microsporidial 16S rRNA gene sequences acquired from the NCBI GenBank database were aligned using the MEGALIGN software package (DNASTAR, Inc., Madison, Wis.). From this alignment, a genetically conserved region of the genus Encephalitozoon was flanked by genetically variable regions with respect to the phylum Microsporidia was chosen as the target site for the probe and primer sets. The sequences of the Encephalitozoon genus-specific fluorescent-labeled probe (EncephP1) and Encephalitozoon hellem (EhelF1/EhelR2), Encephalitozoon cuniculi (EcunF1/EcunR2) and Encephalitozoon intestinalis (EintF1/EintR2) species-specific primer sets were selected using the ABI Primer Express® program (Applied Biosystems) according to the manufacturer's instructions. The nucleic acid sequence of the probe and primer sets were checked against all available nucleic acid sequences in the NCBI GenBank database using the BLAST Search program to confirm, in the former, genus specificity, and in the latter, species specificity. The probe and primers were prepared by Applied Biosystems.

Cyclospora cayetanensis DNA Isolation

One hundred μl of either a flow cytometric or hemacytometer counted Cyclospora cayetanensis oocyst suspension were added to a capped 2 ml conical bottom screw-cap tube containing 0.35 g or autoclaved, acid-washed glass beads (425-600 microns, Sigma-Aldrich Co., St. Louis, Mo.) and 180 μl of lysis buffer supplied with the Dneasy Tissue Kit (Qiagen, Inc., Valencia, Calif.). The oocyst wall was disrupted by agitation in a Mini-Bead Beater (BioSpec Products, Inc., Bartlesville, Okla.) for one minute at maximum speed. The lysate was removed that the genomic DNA was isolated using the Dneasy™ Tissue Kit according to the manufacturer's instructions. The purified DNA was stored at −20° C. until further use.

Enumeration of Microsporidial Spores

Purified spores from each of the three Encephalitozoon species tested were enumerated using either a flow cytometer or a hemacytometer. Flow cytometric counting of microsporidial spores was performed with a FACSVantage SE (Beckton Dickinson) equipped with an argon laser 488 nm, and Clone-Cyt software (Becton Dickinson). FACS Flow isotonic (Becton Dickinson) was used as sheath fluid. The spores were gated and sorted by their forward (FSC) and side (SSC) scatter profile (FSC gain 2 FSC threshold 26V, SSC gain 1 352 V). The spores were sorted into a 1.5 ml microcentrifuge tube containing 0.1 ml reagent water. Hemacytometric counting of microsporidial spores was performed at 400× magnification with a Zeiss Axioskop 20 microscope (Carl Zeiss) equipped with phase-contrast optics. Eight replicate hemacytometer platforms were counted and averaged for each microsporidia species to determine the spore suspension density.

Extraction and Isolation of Microsoporidial DNA

A predetermined number of spores from each of the three Encephalitozoon species tested were flow counted, and known amounts of spores per 100 μl were used for he DNA extraction procedure. A 100 μl suspension of counted microsporidial spores was added to a capped 1 ml conical bottom screw-cap tube containing 0.35 g of autoclaved, acid-washed glass beads (425-600 nM, sigma-Aldrich Co.). The spores were disrupted by agitation in a Mini-Bead Beater (Bio-Spec. Products, Inc.) for one minute at maximum speed. The genomic DNA was isolated using Dneasy™ Tissue Kits, and the purified DNA was stored at −20° C.

Real-Time Quantitative Assay for Cyclospora cayetanensis

PCR was performed in 0.5 ml MicroAmp optical PCR tubes (Applied Biosystems) in a total reaction volume of 25 μl. Each reaction contained 5 μl of extracted Cyclospora cayetanensis DNA, 2.4 μl of a 450 nM solution of CcayP1 (the Taqman probe directed against Cyclospora cayetanensis), 2.4 μl of a 5.2 μM solution of the CcayF1 (forward primer), 2.4 μl of a 5.2 μM solution of the CcayR1 (reverse primer), 0.9 μl of a 2 mg/ml bovine serum albumin fraction V (Sigma-Aldrich Co., St. Louis, Mo.), and 11.9 μl of the 2× Taqman® Universal PCR Master Mix (Applied Biosystems, Foster City, Calif.). This master mix contains proprietary amounts of Amplitaq Gold® DNA polymerase, Amperase® UNG, dNTPs (dCTP, dGTP, DATP, and dUTP), passive reference 6-carboxyX-rhodamine (ROX), MgCl₂ and other buffer components. The optical tubes were inserted into a MicroAmp® 96-well tray/retainer set (Applied Biosystems, Foster City, Calif.) and sealed with MicroAmp® optical caps (applied Biosystems). The 96-well tray containing the samples was centrifuged for five minutes at 500×g before being inserted into an ABI-Prism 7700 Sequence Detector (Applied Biosystems). The thermal cycling conditions included: two minutes at 50° C., 10 minutes at 95° C., followed by 40 cycles of 95° C. for 0.25 minute and 60° C. for one minute. Five replicates were prepared for each sample.

Real-Time Quantitative Assay for Encephalitozoon spp.

PCR was performed in 25 μl reactions using 4-5 pmol of the EncephP1 probe, 10 pmol of each primer, 1.8 μg/μl of bovine serum albumin Fraction V (Sigma-Aldrich Col, St. Louis, Mo.), 1× Taqman® Universal PCR Master Mix (Applied Biosystems, Foster City, Calif.). Master Mix contains proprietary amounts of Amplitaq Gold® DNA polymerase, Amperase® UNG, dNTPs (dCTP, dGTP, dATP, and dUTP), passive reference 6-carboxy-X-rhodamine (ROX), MgCl₂ and other buffer components. The optical tubes were inserted into a MicroAmp® 96-well tray/retainer set (applied Biosystems, Foster City, Calif.) and 5 microliters of extracted microsporidial DNA template. PCR. samples containing reagent water rather than DNA extracts were run as negative controls in each experiment. PCR was performed with an ABI-Prism 7700 Sequence Detector using PCR cycling conditions of: two minutes at 50° C., 10 minutes at 95° C., followed by 40 cycles of 95° C. for 0.25 minute and 60° C. for one minute.

Optimization of Probe and Primer Concentrations for Encephalitozoon spp.

The original probe and primer concentration for each assay was determined by varying the concentration of the EncephP1 probe between 25 nM and 250 nM and the species-specific primer sets EhelF1/EhelR2, EcunF1/EcunR2 and EintF1/EintR2 between 100 nM and 1000 nM.

Specificity Testing for Encephalitozoon spp. Probe and Primer Sets

The species-specificity of each fluorogenic 5′nuclease PCR assay was tested using DNA isolated from 10⁴ microsporidial spores from the other two Encephaliozoon species tested as well as the numerous other protozoa, algae and bacteria listed in Table 1.

TABLE 1
Protozoa, algae and bacteria used for specificity testing
of the fluorogenic 5′ nuclease PCR Encephalitozoon spp. assays
presented in this study

| Organism | Source |
|---|---|
| Encephalitozoon hellem | M. M. Marshall, UAz, Tucson, |
| (CDC:0291:V213) | AZ |
| Encephalitozzon cuniculi (ATCC | |
| #50502) | |
| Encephalitozoon intestinalis 9ATCC | |
| #50603) | |
| Giardia lamblia (CDC:0284) | Originally G. S. Govinda, CDC, |
| | Atlanta; cultivated at U.S. |
| | EPA, Cincinnati, OH |
| Giardia muris | Originally D. P. Stevens, Case |
| | Western Reserve University, |
| | Cleveland, OH; cultivated at |
| | U.S. EPA, Cincinnati, OH |
| Cryptosporidium parvum, H. Moon | Originally C. R. Sterling, |
| Iowa isolate | UAz, Tucson; cultivated at |
| | U.S. EPA, Cincinnati, OH |
| Cryptosporidium muris | Originally M. Iseki, Osaka, |
| | Japan; cultured at U.S. EPA, |
| | Cincinnati, OH |
| Toxoplasma gondii | J. P. Dubey, USDA, Beltsville |
| | Agriculture Research Center, |
| | Beltsville, MD |
| Bacillus subtilis 130740 | R. E. Stetler, U.S. EPA, |
| | Cincinnati, OH |
| Bacillus cereus (ATCC #14579) | M. R. Rodgers, U.S. EPA, |
| Aeromonas 492-BZ | Cincinnati, OH |
| Helicobacter pylori | A. E. McDaniels, U.S. EPA, |
| | Cincinnati, OH |
| Enterococcus faecalis | J. W. SantoDomingo, U.S. EPA, |
| Citrobacter freundii | Cincinnati, OH |
| Klebsiella pneumoniae | |
| Staphylococcus aureus | |
| Enterococcus faecium | |
| Serratia marcescens | |
| Proteus vulgaris | |
| Chlorococcum botryoides | R. E. Stetler, U.S. EPA, |
| Chlorococcum macrostigmatum | Cincinnati, OH |
| Scenedesmus brasiliensis | |

SYBR Green Analysis of Encephalitozoon spp. PCR products

SYBR Green analyses were performed with the same PCR reaction mixture used in the fluorogenic 5′ nuclease PCR assays except that a 1×SYBR Green PCR Buffer (Applied Biosystems) was substituted in place of the EncephP1 probe. The PCR cycling conditions were identical to those used in the fluorogenic 5′ nuclease PCR assay except that a 20 minute disassociation melting protocol (60-95° C.) was performed after the 40 PCR cycles.

Data Analysis for Cyclospora cayetanensis

The fluorescent intensity of the reporter dye was read automatically during the anneal-extension step of PCR using an ABI-Prism 7700 Sequence Detector. The fluorescence data that were generated during PCR were analyzed with the sequence detector software (version 1.6.3, Applied Biosystems) that was loaded onto a Macintosh 4400/200 PowerPC (Apple Computer, Santa Clara, Calif.). The sequence detector monitored the increase in fluorescence that occurred during the anneal-extension step when the fluorescently labeled probe annealed to the specific PCR amplicon and was cleaved by the AmpliTaq Gold ® DNA polymerase. This increase in fluorescence was displayed as a value termed ΔRₙ (Rₙ⁺−Rₙ⁻), where Rₙ⁺ is the emission intensity of the reported dye divided by the emission intensity of the internal passive reference dye, and Rₙ⁻ is the emission intensity during early PCR cycles divided by the emission intensity of the internal passive reference dye. The passive reference dye in the Taqman® Universal PCR Master Mix normalizes for differences in the emission intensities that can occur from fluctuations caused by changes in the PCR sample volume or concentration. Furthermore, the sequence detector software calculates a threshold value (background noise level) for each assay that is more than 10 standard deviations above the mean background of the fluorescence data collected in cycles 3 through 15 of the assay before any significant increase in fluorescence has occurred within the sample. The PCR cycle at which the increase in fluorescence (ΔRₙ) of a sample exceeds the background noise is called the threshold cycle (C_{T}). The data acquired during PCR by the sequence detector was exported and regenerated in Microsoft Excel (Microsoft Corporation, Redman, Wash.), where calibration curves were constructed for each of the three microsporidial species using plots of C_{T} VS. logarithm of the spore count in the PCR assay.

Optimization of Probe and Primer Concentrations for Cyclopspora cayetanensis

The concentrations of the Taqman primers and probe were varied to achieve the highest level of sensitivity and specificity in the real-time PCR assay. Probe concentrations ranging from 40 nM to 1000 nM per reaction were tested to determine optimal conditions for this real-time PCR assay.

Specificity Testing for Cyclospora cayetanensis

The specificity of the optimized real-time PCR assay was tested with the protozoa, algae, and bacteria listed in Table 2.

TABLE 2
Protozoa, algae and bacteria used for specificity testing
of the real-time PCR assay for Cyclospora cayetanensis

| Organism | Source |
|---|---|
| Eimeria mieshulzi | D. W. Duszynski, UNM, |
| Eimeria falcifarum | Albuquerque, NM |
| Eimeria tenella | S. Harris, U.S. EPA, Manchester, |
| | WA |
| Cyclospora colobi | M. L. Eberhard, CDC, Atlanta, GA |
| Encephalitozoon hellem | M. M. Marshall, UAz, Tucson, AZ |
| (CDC:0291:V213) | |
| Encephalitozoon cuniculi (ATCC | |
| #50502) | |
| Encephalitozoon intestinalis (ATCC | |
| #50603) | |
| Giardia lamblia (CDC:0284) | Originally G. S. Govinda, CDC, |
| | Atlanta; cultivated at U.S. EPA, |
| | Cincinnati, OH |
| Giardia muris | Originally D. P. Stevens, Case |
| | Western Reserve University, |
| | Cleveland, OH; cultivated at U.S. |
| | EPA, Cincinnati, OH |
| Cryptosporidium parvum, H. Moon | Originally C. R. Sterling, UAz, |
| Iowa isolate | Tucson, AZ; cultivated at U.S. |
| | EPA, Cincinnati, OH |
| Cryptosporidium muris | Originally M. Iseki, Osaka City |
| | University Medical School, Osaka, |
| | Japan; cultured at U.S. EPA, |
| | Cincinnati, OH |
| Toxoplasma gondii | J. P. Dubey, USDA, Belstville |
| | Agriculture Research Center, |
| | Beltsville, MD |
| Bacillus subtilis 130740 | R. E. Stetler, U.S. EPA, |
| | Cincinnati, OH |
| Bacillus cereus (ATCC #14579) | M. R. Rodgers, U.S. EPA, |
| Aeromonas 492-BZ | Cincinnati, OH |
| Helicobacter pylori | A. E. McDaniels, U.S. EPA, |
| | Cincinnati, OH |
| Enterococcus faecalis | J. W. SantoDomingo, U.S. EPA, |
| Citrobacter freundii | Cincinnati, OH |
| Klebsiella pneumoniae | |
| Staphylococcus aureus | |
| Enterococcus faecium | |
| Serratia marcescens | |
| Proteus vulgaris | |
| Chlorococcum botryoides | R. E. Steler, U.S. EPA, |
| Chlorococcum macrostigmatum | Cincinnati, OH |
| Scenedesmus brasiliensis | |

Controls for Cyclospora cayetanensis

PCR samples containing reagent water instead of DNA extracts were run as negative controls in each 96-well plate. Calibration Curve Generation and Testing

A calibration curve was generated from serial dilutions of Cyclospora cayetanensis DNA, ranging from 10³ to 10⁰ oocysts. The serial dilution of Cyclospora cayetanensis DNA was performed in duplicate using two DNA extracts that were processed identically. Each sample in the two DNA dilution series (10³, 10², 10¹ and 10⁰) was analyzed in five separate PCR samples. Thus each data point on the calibration curve is the average of ten PCR samples. This calibration curve was evaluated with flow cytometric counted of Cyclospora cayetanensis oocysts.

For the Encephalitozoon species tested, calibration curves were generated using serial ten-fold diluted DNA that was originally extracted from 8×10⁵ hemacytometer counted spores from each of the three Encephalitozoon species. Five microliters of the serial ten-fold diluted DNA extract containing DNA corresponding to 10⁴, 10³, 10², 10¹, 10⁰ or 10⁻¹ Encephalitozoon hellem, Encephalitozoon cuniculi, or intestinalis spores was added to the respective PCR sample. DNA extraction procedures were performed in triplicate for each microsporidial species in order to evaluate the reproducibility of the DNA isolation technique. Each data point within a calibration curve corresponds to the mean CT value of 15 replicates, five from each of the three individual DNA extraction procedures.

Validation of Calibration Curves for Encephalitozoon spp.

DNA isolated from 8000, 800, 80 and 8 flow cytometric enumerated spores from each of the three Encephalitozoon species tested was used to validate the calibration curves. Three independent DNA extractions were performed for each of the spore counts listed above. Five microliters of each DNA extract listed above was added to PCR sample so that DNA corresponding to 10², 10¹, 10⁰ or 10⁻¹ Encephalitozoon hellem, Encephalitozoon cuniculi, or Encephalitozoon intestinalis spores was present within a reaction mixture. Each data point used to validate the calibration curves corresponds to the mean CT value of 15 replicates, five from each of the three individual DNA extraction procedures.

Data Analyses for Encephalitozoon spp.

The real-time fluorescence data generated during the fluorogenic 5′ nuclease PCR assays was analyzed with sequence detector software (version 1.6.3; Applied Biosystems) on a Macintosh 4400/200 PowerPC (Apple Computer, Santa Clara, Calif.). The sequence detector monitors the increase in fluorescence that occurs when the fluorescent-labeled probe anneals to the specific PCR; amplicon and is cleaved by the DNA polymerase during the anneal-extension step. This fluorescence increase is expressed as ΔRₙ (Rₙ⁺−Rₙ⁻), where Rₙ⁺ is the emission intensity of the reporter dye divided by the emission intensity of the passive reference, and Rₙ⁻ is the emission intensity during early PCR cycles divided by the emission intensity of the reference. The reference in the Taqman ® Universal PCR Master Mix normalizes for differences in the emission intensities that can occur due to fluctuations caused by changes in the PCR sample volume or concentration. Furthermore, the sequence detector software calculates a threshold value (background noise level) for each assay that is more than 10 standard deviations above the mean background of the fluorescence data collected in cycles 3 through 15 of the assay before any significant increase in fluorescence has occurred within the sample. The PCR cycle at which the increase in fluorescence (ΔRₙ) of a sample exceeds the background noise is called the threshold cycle (C_{T}). The C_{T} is inversely proportional to the copy number of the target template. The higher the template concentration, the lower the threshold cycle measured. C_{T} and ΔRₙ values were exported and regenerated in Microsoft Excel (Microsoft Corporation, Redman, Wash.).

Results for Cyclospora cayetanensis

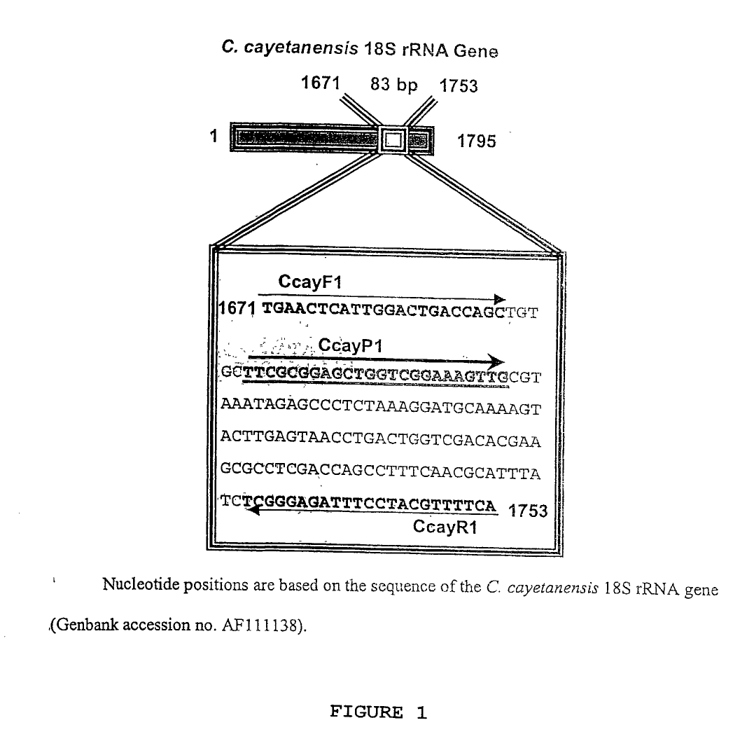

The nucleic acid sequences of the of Cyclospora cayetanensis-specific Taqman probe and primer set are listed in FIG. 1. Furthermore, the relative position of this Taqman probe and primer set with respect to the 18S rRNA gene sequence of Cyclospora cayetanensis also is shown in FIG. 1. When the sequence of the of Cyclospora cayetanensis-specific Taqman probe and primer set were checked against all available nucleic acid sequences in the NCBI GenBank database, this search revealed that the Taqman probe and primer set were specific to of Cyclospora cayetanensis. This was corroborated when the specificity of the real-time PCR assay was tested using DNA extracted from numerous protozoa, bacteria, and algae. Analysis of these non-specific samples resulted in CT values of 40 in this of Cyclospora cayetanensis-specific real-time PCR assay.

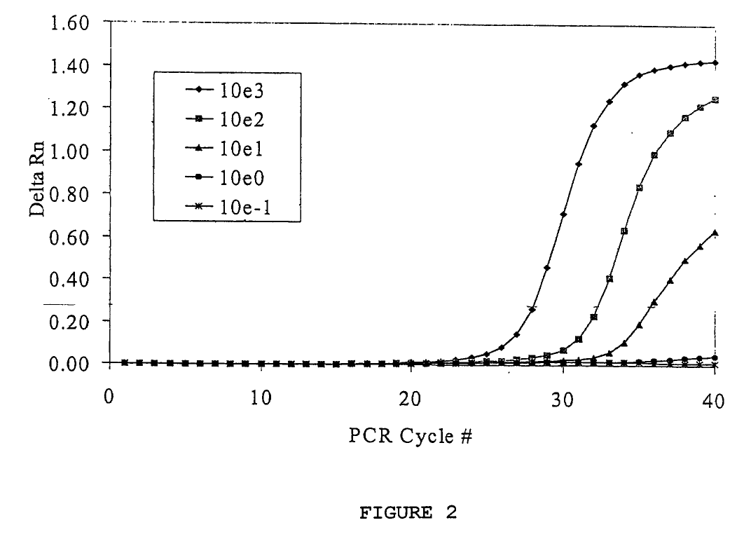

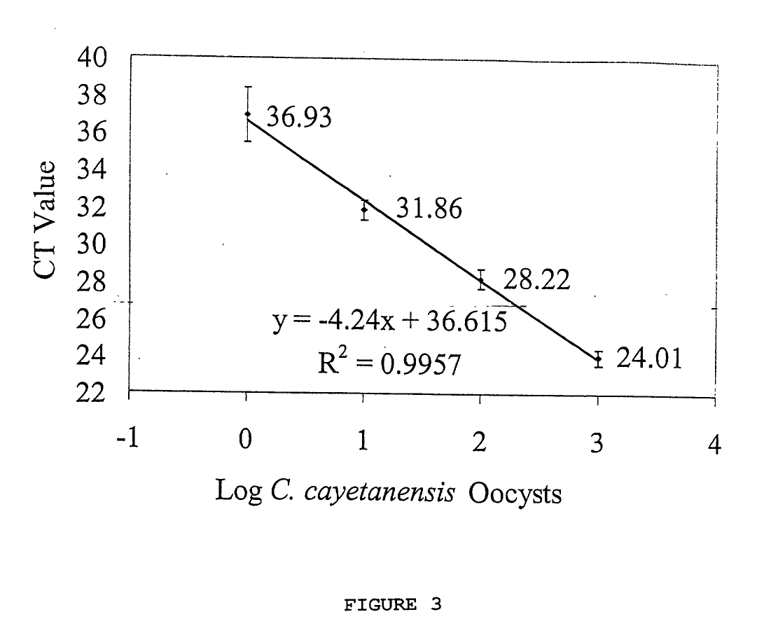

A typical amplification plot (ΔR vs. PCR cycle) constructed from fluorescence data generated from DNA corresponding to 10³ to 10⁻¹ of Cyclospora cayetanensis oocysts is shown in FIG. 2. A significant increase in fluorescence is measurable to 100 oocysts, while 10⁻¹ oocyst was indistinguishable from a no template containing negative control sample. The calibration curve generated from serial dilutions of Cyclospora cayetanensis DNA, ranging from 10³-10⁻¹ oocysts is shown in FIG. 3. The linear range of this real-time PCR assay was 4 logs with a correlation coefficient of 0.0057. The sensitivity and intra-experimental variability of this assay with respect to the DNA dilution series data is shown Table 3.

TABLE 3
Sensitivity and intra-experimental variability of a real-
time PCR assay for Cyclospora cayetanensis

| C. cayetanesis | | | | |
|---|---|---|---|---|
| Oocyst Count by | | | Standard | % Coefficient |
| Hemacytometer | N | Mean C_{T} | Deviation | of Variation |
| 1000 | 10 | 24.10 | .43 | 1.78 |
| 100 | 10 | 28.22 | .53 | 1.88 |
| 10 | 10 | 31.86 | .55 | 1.73 |
| 1 | 10 | 36.93 | 1.45 | 3.92 |

Flow cytometric counted of Cyclospora cayetanensis oocysts were used to verify whether the calibration curve could be used for future quantitation of unknown numbers of oocysts, as shown in Table 4.

TABLE 4
Evaluation of the calibration curve with known numbers of
flow cytometric counted Cyclospora cayetanensis oocysts

| | | | | | ΔC_{T}^{b} |
|---|---|---|---|---|---|
| Oocyst | | Mean C_{T}^{a} | Standard | Expected C_{T} | (Experimental |
| Count | N | Value | Deviation | Value | Expected) |
| 100 | 5 | 27.89 | 0.338 | 28.22 | 0.34 |
| 100 | 5 | 28.63 | 0.130 | 28.22 | 0.41 |
| 100 | 5 | 27.51 | 0.385 | 28.22 | 0.71 |
| 10 | 5 | 32.44 | 0.368 | 31.86 | 0.58 |
| 10 | 5 | 31.80 | 0.193 | 31.86 | 0.06 |
| 10 | 5 | 31.36 | .0595 | 31.86 | 0.50 |
| 1 | 5 | 37.28 | 2.145 | 36.93 | 0.35 |
| 1 | 5 | 39.11 | 1.660 | 36.93 | 2.18 |
| 1 | 5 | 37.57 | 2.000 | 36.93 | 0.64 |

^{a}C_{T} is the cycle number at which the change in fluorescence surpasses the threshold, calculated as the average change in fluorescence during PCR cycles three to 15.
^{b}ΔC_{T} is the change in C_{T} value from the value calculated on the basis of the standard curve to the value observed from flow cytometry enumerated oocysts.

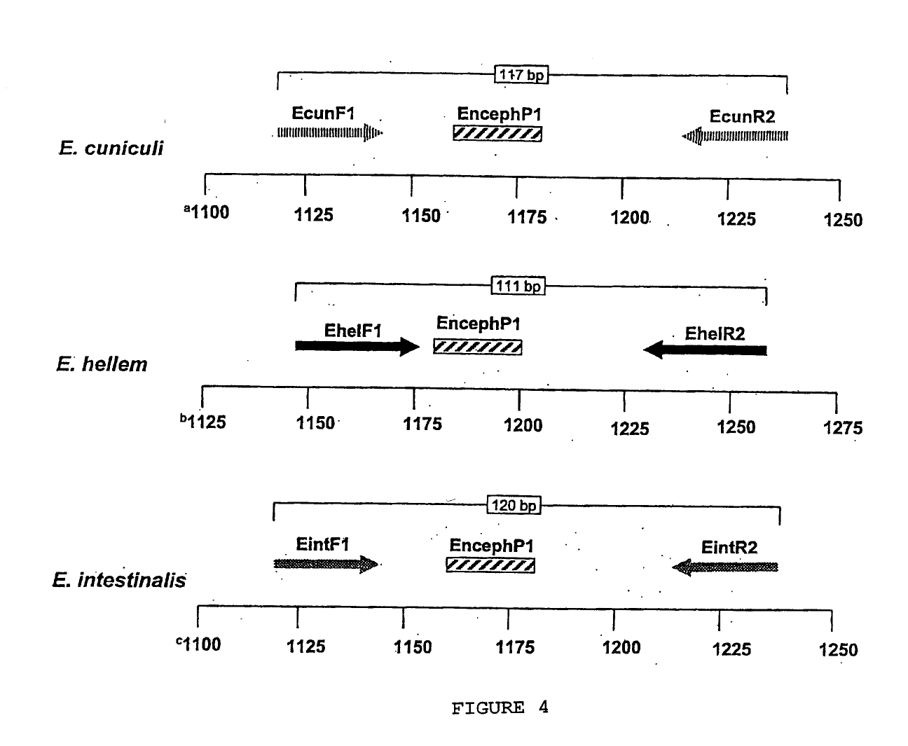

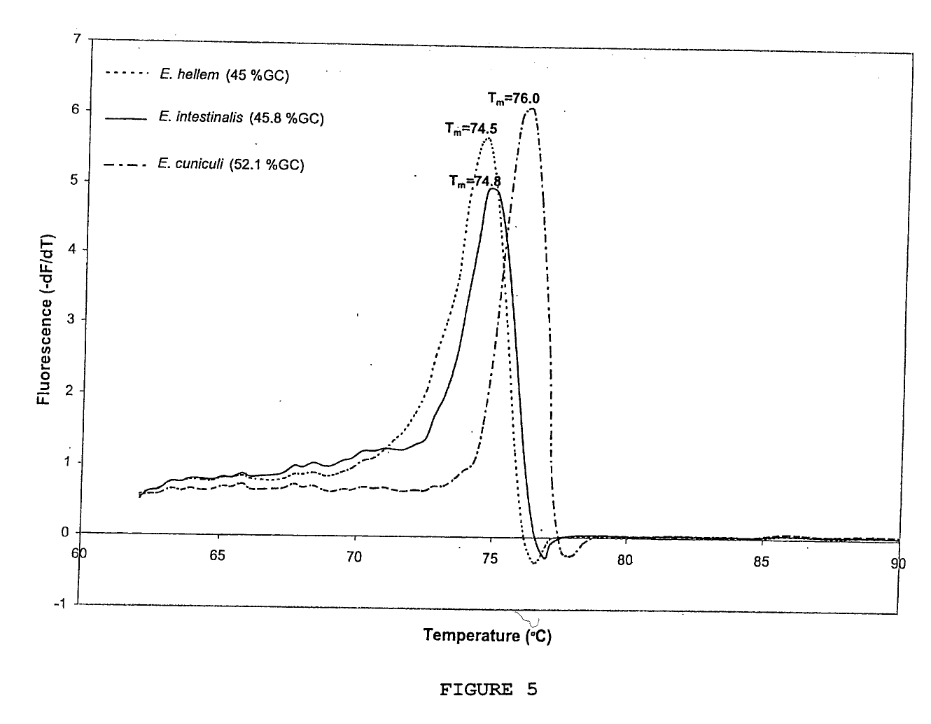

The nucleic acid sequences of the Encephalitozoon-specific probe and Encephalitozoon hellem, Encephalitozoon cuniculi, and Encephalitozoon intestinalis-specific primer sets used in the study described above are shown in Table 5. When the genus-specific probe and the species-specific primer sets were checked against all available nucleic acid sequences in the NCBI GenBank database using he NCBI Blast Search program, the search revealed that the EncepthP1 probe was specific for the genius Encephalitozoon and the primer sets EhelF1/EhelR2, EcunF1/EcunR2 and EintF1/EintR2 were unique to their respective Encephalitozoon species. The relative position of the genus-specific probe and species-specific primer sets within the 16S rRNA gene sequences of all three Encephalitozoon species is shown in FIG. 4. Aqarose gel electrophoresis verified that amplicons of 117, 111, and 120 bp in length were produced with DNA isolated from Encephalitozoon cuniculi, Encephalitozoon hellem, and Encephalitozoon intestinalis, respectively (data not shown). As shown in FIG. 5, the Encephalitozoon cuniculi, Encephalitozoon hellem and Encephalitozoon intestinalis-specific assays produced amplicons that had characteristic melting temperature of 76, 74.5 and 74.8° C. that concur with their respective base composition.

TABLE 5
Encephalitozoon spp. Oligonucleotides used in the fluorogenic 5′ nuclease PCR assays

| Probe or | | Genome | Length | Melting Temp |
|---|---|---|---|---|
| Primer | Seqence (5′-3′) | Localization | (Nucleotides) | (° C.)^{d} |
| EncephP1 | 6FAM-CCC TGT CCT TTG TAC ACA CCG CCC-TAMRA | 1157-1180^{a} | 24 | 68 |
| | | 1177-1200^{b} | 24 | 68 |
| | | 1158-1181^{c} | 24 | 68 |
| EcunF1 | TCC TAG TAA TAG CGG CTG ACG AA | 1118-1140^{a} | 23 | 59 |
| EcunR2 | ACT CAG GAC TCA GAC CTT CCG A | 1234-1213^{a} | 22 | 59 |
| EhelF1 | GAA TGA TTG AAC AAG TTA TTT TGA ATG TG | 1147-1175^{b} | 29 | 59 |
| EhelR2 | AAC ACG AAA GAC TCA GAC CTC TCA | 1257-1234^{b} | 24 | 58 |
| EintF1 | AAT TCC TAG TAA TAA CGA TTG AAC AAG TTG | 1116-1145^{c} | 30 | 59 |
| EintR2 | ACG AAG GAC TCA GAC CTT CCA A | 1235-1214^{c} | 22 | 59 |

^{a}Nucleotide positions are based on the E. cuniculi 16S rRNA gene sequence (Genbank accession no. X98469).
^{b}Nucleotide positions are based on the E. hellem 16S rRNA gene sequence (Genbank accession no. L39108).
^{c}Nucleotide positions are based on the E. intestinalis 16S rRNA gene sequence (Genbank accession no. U09929).
^{d}Tₘ calculated using Primer Express software
6Fam-6-carboxyfluorescein
TAMRA-6-carboxytetramethyl-rhodamine

Generation of Calibration Curves for Encephalitozoon spp.

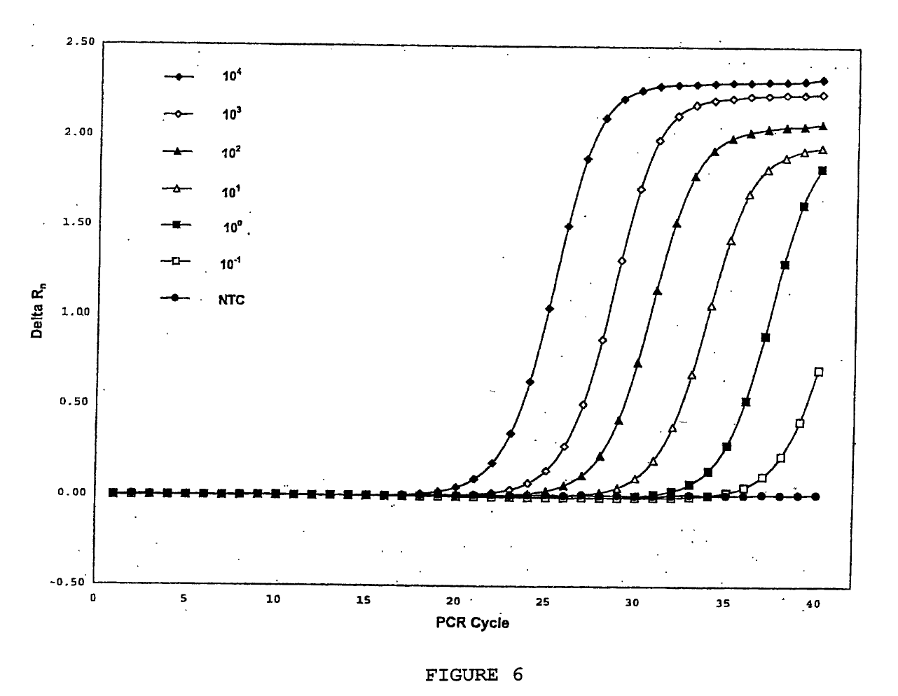

FIG. 6 shows the sensitivity of the fluorogenic 5′ nuclease PCR assays in a representative amplification plot (ΔRₙ vs. PCR cycle) produced using serial ten-fold dilutions of DNA corresponding to 10⁴-10⁻¹ Encephalitozoon hellem spores per PCR sample. The non-template control (NTC) did not produce any detectable fluorescence signal above background, whereas a significant increase in fluorescence was observed with DNA that would theoretically correspond to 10⁻¹ spores. Calibration curves obtained with serial ten-fold dilutions of genomic DNA that was isolated from hemacytometer enumerated spores from each of the three Encephalitozoon species tested are shown in FIGS. 7a-7c. The CT values for the Encephalitozoon cuniculi, Encephalitozoon hellem and Encephalitozoon intestinalis-specific calibration curves, standard deviation, and the intra-experimental variability of the fluorogenic 5′ nuclease PCR assays are reported in Table 6. The linear range of each species-specific fluorogenic 5′ nuclease PCR assay analyzed here in 5 logs with correlation coefficients of 0.9998, 0.9995 and 0.9996 for Encephalitozoon cuniculi, Encephalitozoon hellem and Encephalitozoon intestinalis, respectively. The slopes and PCR amplification efficiencies of each species-specific fluorogenic 5′ nuclease PCR assay are give in FIGS. 7a-7c.

TABLE 6
Sensitivity and intra-experimental variability of
hemocytometric data used to generate the calibration curves in the
fluorogenic 5′ nuclease PCR assays for Encephalitozoon spp.

| Spore Count in | Mean C_{T} ± Stdv | % Coefficient |
|---|---|---|
| Assay | (N = 15) | of Variation |
| E. cuniculi | | |
| 10⁴ | 17.82 ± 0.16 | 0.90 |
| 10³ | 21.12 ± 0.20 | 0.95 |
| 10² | 24.77 ± 0.27 | 1.09 |
| 10¹ | 28.41 ± 0.13 | 0.46 |
| 10⁰ | 31.88 ± 0.66 | 2.08 |
| 10⁻¹ | 35.53 ± 0.81 | 2.07 |
| E. hellem | | |
| 10⁴ | 19.19 ± 0.33 | 1.72 |
| 10³ | 22.43 ± 0.17 | 0.76 |
| 10² | 26.11 ± 0.25 | 0.96 |
| 10¹ | 29.94 ± 0.26 | 0.87 |
| 10⁰ | 33.46 ± 0.66 | 1.97 |
| 10⁻¹ | 33.83 ± 1.90 | 5.16 |
| E. intestinalis | | |
| 10⁴ | 18.16 ± 0.13 | 0.72 |
| 10³ | 21.67 ± 0.18 | 0.83 |
| 10² | 25.24 ± 0.22 | 0.87 |
| 10¹ | 29.00 ± 0.17 | 0.59 |
| 10⁰ | 32.64 ± 0.69 | 2.11 |
| 10⁻¹ | 36.65 ± 1.67 | 4.56 |

Testing the Quantitative Capability of the Species-Specific Calibration Curve for Encephalitozoon spp.

When DNA isolated from flow cytometric enumerated spores from each Encephalitozoon species was compared to the appropriate species-specific calibration curve, 94% of all samples were within two standard deviations of the predicted value, as shown in Table 7.

TABLE 7
Evaluation of the respective species-specific
Encephalitozoon calibration curves with flow cytometric counted
spores

| Spore Count in Assay | Mean C_{T} ± Stdv (N = 15) |
|---|---|
| E. cuniculi | |
| 10² | 24.65 ± 0.27 |
| | 24.33 ± 0.26 |
| | 24.67 ± 0.18 |
| 10¹ | 28.02 ± 0.31 |
| | 28.51 ± 0.22 |
| | 28.13 ± 0.17 |
| 10⁰ | 31.52 ± 0.56 |
| | 30.75 ± 0.39 |
| | 31.93 ± 0.25 |
| 10⁻¹ | 24.47 ± 0.74 |
| | 34.92 ± 0.52 |
| | 35.38 ± 0.68 |
| E. hellem | |
| 10² | 25.67 ± 0.40 |
| | 25.60 ± 0.32 |
| | 25.69 ± 0.12 |
| 10¹ | 28.79 ± 0.22 |
| | 28.87 ± 0.19 |
| | 29.20 ± 0.42 |
| 10⁰ | 33.06 ± 0.44 |
| | 32.14 ± 0.13 |
| | 32.10 ± 0.20 |
| 10⁻¹ | 35.71 ± 0.85 |
| | 36.60 ± 1.92 |
| | 37.13 ± 1.64 |
| E. intestinalis | |
| 10² | 25.33 ± 0.23 |
| | 25.05 ± 0.16 |
| | 25.46 ± 0.27 |
| 10¹ | 28.83 ± 0.33 |
| | 28.60 ± 0.23 |
| | 28.86 ± 0.15 |
| 10⁰ | 31.61 ± 0.75 |
| | 31.65 ± 0.41 |
| | 31.99 ± 0.39 |
| 10⁻¹ | 36.25 ± 2.17 |
| | 37.47 ± 2.48 |
| | 35.72 ± 0.77 |

Species-Specificity of the Encephalitozoon Assays

The species-specificity of each fluorogenic 5′ nuclease PCR assay was confirmed using DNA corresponding to 10⁴ spores from each of the three Encephalitozoon species as well as DNA extracted from numerous other protozoa, algae, and bacteria, as shown in Table 1. All samples that did not contain DNA specific for the respective fluorogenic 5′ nuclease PCR assay displayed no increase above background and had CT values of 40.

The present invention provides a technique for detecting human-pathogenic microsporidia to the species level in both clinical and environmental samples which is rapid, sensitive, and scaleable.

The present invention uses the 5′ exonuclease activity of polymerase to cleave a species-specific DNA hybridization probe. This DNA hybridization probe anneals between a set of species-specific primers during the anneal-extension step of PCR. For simplicity as well as cost-effectiveness, the assay of the present invention takes full advantage of a stretch of genetic material in the microsporidial rRNA gene that contains a conserved region for a genus-specific fluorogenic probe, as well as variable flanking regions for species-specific primer sets. The assay design permits the probe to be used either with one set of primers for species-level determination, or with a combination of primer sets for genus-level screening of samples.

The assay of the present invention is sensitive, specific, and resistant to cross contamination. The sensitivity of the assay is conferred by the sensitivity of the nature of the PCR reaction and the sensitivity of the optical detection system. This type of assay permits two levels of selection for specificity, one from the selection of the probe, the other from the selection of appropriate primers. This dual specificity may help eliminate the need for nested PCR reactions for these types of organisms. Resistance to cross contamination is conferred by the two separate systems, as well. First is the incorporation of UNG in the reaction mixture, and the use of UTP as a nucleotide. Once PCR products are synthesized incorporating UTP, the presence of UNG allows for degradation of any spurious PCR product or extraneous RNA contamination. Further, the assay is read as the PCR reaction is carried out, and there is never a need to open the reaction vessel. This latter fact alone greatly limits the potential for cross contamination.

Although microscopy provides a more precise quantitation of the number of oocysts or spores in a sample, the PCR technique improves upon microscopy by allowing re-sampling the microscopy cannot, and comparison of the results of serially diluted PCR samples.

The assays of the present invention not only can provide clinicians and environmental researchers with the identity of a particular microsporidial pathogen which causes a disorder or outbreak, but they can also assist both pharmaceutical companies and physicians in evaluating the efficacy of a drug or treatment regimen for microsporidial infections.

All references cited herein are incorporated by reference.

The foregoing description of the specific embodiments will so fully reveal the general nature of the invention that others can, by applying current knowledge, readily modify and/or adapt for various applications such specific embodiments without departing from the generic concept, and, therefore, such adaptions and modifications should and are intended to be comprehended within the meaning and range of equivalents of the disclosed embodiments. It is to be understood that the phraseology or terminology employed herein is for the purpose of description and not of limitation.

What is claimed is:

1. A method for detecting microsporidial species in samples comprising:
a. degrading a dual-fluorescently labeled DNA hybridization probe and a set of species-specific primers during PCR amplification of regions in a gene located within the spores of interest; and
b. reading the fluorescent labels during the PCR process.

2. The method according to claim 1 wherein the microsporidial species is selected from the group consisting of Encephalitozoon hellen, Encephalitozoon, cuniculi, and Encephalitozoon intestinalis.

3. The method according to claim 2 wherein the gene is the 16S rRNA gene sequence of the Encephalitozoon species.

4. The method according to claim 1 wherein the dual-fluorescently labeled probe is labeled with a reporter fluorescent dye and a quencher fluorescent dye.

5. The method according to claim 2 wherein the dual-fluorescently labeled probe is labeled with a reporter fluorescent dye and a quencher fluorescent dye.

6. The method according to claim 3 wherein the dual-fluorescently labeled probe is labeled with a reporter fluorescent dye and a quencher fluorescent dye.

7. A method for detecting Cyclospora cayetanensis in samples comprising:
a. degrading a dual-fluorescently labeled species specific DNA hybridization probe and a primer set during PCR amplification of regions in a gene located within oocysts of interest; and
b. reading the fluorescent labeled during the PCR process.

8. The method according to claim 7 wherein the gene is the 18S rRNA sequence of Cyclospora cayetanensis.

9. The method according to claim 7 wherein the dual-fluorescently labeled probe is labeled with a reporter fluorescent dye and a quencher fluorescent dye.

10. The method according to claim 8 wherein the dual-fluorescently labeled probe is labeled with a reporter fluorescent dye and a quencher fluorescent dye.
